(19) United States
(12) Patent Application Publication
(10) Pub. No.: US 2025/0332375 A1
(43) Pub. Date: Oct. 30, 2025

(54) DEVICES AND METHODS FOR COMFORTING A PATIENT

(71) Applicant: LOMA LINDA UNIVERSITY, Loma Linda, CA (US)

(72) Inventor: Samuel Clough, Loma Linda, CA (US)

(21) Appl. No.: 19/189,497

(22) Filed: Apr. 25, 2025

Related U.S. Application Data
(60) Provisional application No. 63/638,911, filed on Apr. 25, 2024.

(51) Int. Cl. A61M 21/02; A61M 21/00

(52) U.S. Cl. CPC A61M 21/02; A61M 2021/0022; A61M 2240/00

(57) ABSTRACT

Systems and methods are described herein for providing a soothing simulated patting treatment to patients, including infants and neonates. One example is a patient soothing system that includes a patting device configured to be pneumatically powered to rapidly inflate and deflate to provide a simulated patting force to a patient. The system includes a control module having control circuitry and an air pulse regulator with at least one pneumatic solenoid valve that is fluidly connected to the patting device via a flexible tube. The control circuitry is configured to actuate the at least one pneumatic solenoid valve of the air pulse regulator to provide rapid inflation and deflation pulses of air to rapidly inflate and deflate the patting device, thereby to yield the simulated patting force.

### CROSS REFERENCE TO RELATED APPLICATIONS

This application claims priority to, and the benefit of U.S. Provisional Application No. 63/638,911, filed Apr. 25, 2024, titled “DEVICES AND METHODS FOR COMFORTING AN INFANT,” the disclosure of which is incorporated herein by reference in its entirety.

### TECHNICAL FIELD

This disclosure relates generally to systems and methods of providing comfort to a patient or user. More specifically, the disclosure relates to a systems and methods capable of introducing a simulated patting treatment protocol to a patient, such as an infant in a hospital or neonatal intensive care unit (ICU).

### BACKGROUND

Generally, newborn infants or small children may be afflicted with different distressing ailments, such as, for example, colic, acid reflux, drug withdrawal, digestive issues, difficulty eating or sleeping, transitioning from the intrauterine environment, and so forth. In some cases, the infants may be subject to these conditions for extended periods of time, which may include extended periods of crying. There are limited ways of easing the child's discomfort. Frequently, the child may be hugged or swaddled, along with receiving pats or rubbing to their back sides. However, for infants in, for example, a neonatal intensive care unit, the child may be suffering from a condition that requires comforting for excessive periods of time, and nurses are often limited in the amount of time they can spend comforting the child.

### SUMMARY

With the foregoing in mind, Applicant has developed systems and methods for providing a soothing simulated patting treatment to patients, including infants and neonates. One embodiment of such a system is a patient soothing system that includes a patting device configured to be pneumatically powered to rapidly inflate and deflate to provide a simulated patting force to a patient. The system has a control module including control circuitry and an air pulse regulator with at least one pneumatic solenoid valve that is fluidly connected to the patting device via a flexible tube. The control circuitry is configured to actuate the at least one pneumatic solenoid valve of the air pulse regulator to provide rapid inflation and deflation pulses of air to rapidly inflate and deflate the patting device, thereby to yield the simulated patting force.

In some embodiments, the patient is an infant, and the patient soothing system further includes a blanket having a pocket configured to receive the patting device, in which the blanket is configured to wrap around a torso of the infant to secure the patting device at a posterior side of the infant. In some embodiments, the blanket includes one or more additional pockets configured to receive a disposable warming pack or a disposable cooling pack. In some embodiments, the blanket is translucent to light at wavelengths ranging from about 460 nanometers (nm) to about 490 nm.

In some embodiments, the patting device includes an air bladder formed from excessive density polyethylene (EDPE), high-density polyethylene (HDPE), silicone, or any combination thereof. In some embodiments, the patting device is waterproof and configured to be handwashed with soap and water for reuse with another patient. In some embodiments, the patting device has a width of about 10 centimeters (cm), a length of about 15 cm, an inflated thickness of about 2.5 cm, and a deflated thickness about 1 cm or less.

In some embodiments, the control circuitry includes a microcontroller communicatively connected to one or more metal-oxide-semiconductor field-effect transistors (MOSFETs). The microcontroller is configured to provide control signals to switch the MOSFETs between an on-state and an off-state, and the MOSFETs is operably coupled to actuate the at least one pneumatic solenoid valve between operational states to rapidly inflate and deflate the patting device. In some embodiments, an air outlet of the air pulse regulator is fluidly connected to the patting device, an air inlet of the air pulse regulator is fluidly connected to an air source, and an air exhaust outlet of the air pulse regulator is open to ambient air pressure. In some embodiments, the air source includes an air supply of a medical treatment facility or vehicle, a compressed air cylinder, or an external air pump. In some embodiments, the control module includes an internal air pump, and the air source is the internal air pump. In some embodiments, the at least one pneumatic solenoid valve includes a 3/2-way pneumatic solenoid valve, and the 3/2-way pneumatic solenoid valve is fluidly connected to the air outlet, the air inlet, and the air exhaust outlet of the air pulse regulator. In some embodiments, the at least one pneumatic solenoid valve includes a first 2/2-way pneumatic solenoid valve and a second 2/2-way pneumatic solenoid valve, and the first 2/2-way pneumatic solenoid valve is fluidly connected to the air outlet and the air inlet of the air pulse regulator, and the second 2/2-way pneumatic solenoid valve is fluidly connected to the air outlet and the air exhaust outlet of the air pulse regulator.

In some embodiments, the control module includes power conversion circuitry configured to receive and convert alternating current (AC) mains electrical energy into direct current (DC) electrical energy to power at least the control circuitry and the at least one pneumatic solenoid valve of the air pulse regulator during operation. In some embodiments, the control module includes an on-board battery configured to provide DC electrical energy to power at least the control circuitry and the at least one pneumatic solenoid valve of the air pulse regulator during operation. In some embodiments, the simulated patting force is about 30 Newtons (N). In some embodiments, the simulated patting force has a patting rate ranging from about 1 hertz (Hz) to about 3 Hz. In some embodiments, the each of the inflation pulses of air and each of the deflation pulses of air have an air pulse duration ranging from about 650 milliseconds (ms) to about 275 ms.

In some embodiments, the control module includes a user interface configured to receive user inputs to configure parameters of a patting treatment, and the parameters of the patting treatment include a patting rate, a patting force, or a patting treatment duration, or any combination thereof. In some embodiments, the user interface includes a plurality of buttons and a plurality of indicator light emitting diodes (LEDs), in which the plurality of buttons is configured to receive the user inputs to define the parameters of the patting treatment, and the plurality of indicator LEDs is configured to selectively illuminate to indicate the parameters of the patting treatment.

One embodiment of such a method is a method of using a patient soothing system. The method includes fluidly connecting a control module of the patient soothing system to a patting device of the patient soothing system via a flexible tube. The method includes placing the patting device in indirect contact with a patient. The method includes activating the patting device via a user interface of the control module to initiate a patting treatment in which the patting device rapidly inflates and deflates to provide a simulated patting force to the patient.

In some embodiments, the method includes deactivating the patting device via the user interface of the control module to cease the patting treatment. In some embodiments, after conclusion of the patting treatment, the method includes removing the patting device from indirect contact with the patient, disconnecting the control module from the patting device, and washing and drying the patting device for reuse. In some embodiments, the patient is an infant, and the placing of the patting device in indirect contact with the patient includes inserting the patting device into a pocket of a blanket, and wrapping the infant with the blanket such that the patting device is positioned at a posterior side of the patient. In some embodiments, the method includes inserting a warming pack or a cooling pack into a second pocket of the blanket.

In some embodiments, prior to activating the patting device, the method includes selecting one or more patting treatment parameters for the patting treatment via the user interface of the control module, and the patting treatment parameters include a strength of the simulated patting force, a patting rate, or a patting treatment duration, or any combination thereof. In some embodiments, the providing of the simulated patting force to the patient includes repeatedly: (i) actuating at least one pneumatic solenoid valve of the patient soothing system into a first operational position to inflate the patting device, and (ii) after a predefined air pulse duration, actuating the at least one pneumatic solenoid valve of the patient soothing system into a second operational position to deflate the patting device.

In some embodiments, the simulated patting force is about 30 N. In some embodiments, the simulated patting force has a patting rate ranging from about 1 Hz to about 3 Hz. In some embodiments, the patting treatment has a patting treatment duration that ranges from about 10 minutes to about 30 minutes.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1A is a diagrammatic representation of an example of a patient soothing system, according to an embodiment.

FIG. 1B is a diagrammatic representation of another example of a patient soothing system, according to an embodiment.

FIG. 2 is a diagrammatic representation of an example of a patting device of the patient soothing system in an inflated state and in a deflated state, according to an embodiment.

FIG. 3 is a diagrammatic representation of air flow for an example of the patient soothing system during operation, according to an embodiment.

FIG. 4 is a diagrammatic representation of air flow for another example of the patient soothing system during operation, according to an embodiment.

FIG. 5 is a diagrammatic representation of an embodiment of a method of using the patient soothing system, according to an embodiment.

FIG. 6 is a diagrammatic representation of experimental data determined during testing of an example prototype of the patient soothing system, according to an embodiment.

### DETAILED DESCRIPTION

The present disclosure describes various embodiments related to systems and methods for using a pneumatically-powered patting device of a patient soothing system to comfort a patient or user. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “plurality” as used herein refers to two or more items or components. The terms “about” or “approximately” are defined as being close to as understood by one of ordinary skill in the art. In one non-limiting embodiment, these terms are defined to be within 10%, preferably within 5%, more preferably within 1%, and most preferably within 0.5%. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.”

Present embodiments are directed to a patient soothing system capable of soothing patients, such as distressed or crying infants and small children while the caretakers are otherwise occupied. The system is capable of providing patting-like stimulation, heating/cooling, or a combination of patting-like stimulation and heating/cooling. In some embodiments, the system includes a blanket with pockets capable of receiving one or more heating packs or cooling packs, one or more patting devices, or a combination of heating/cooling packs and patting devices. The patting device is generally designed to quickly inflate and deflate to provide the desired force on the patient or user, which is intended to resemble the feeling of a human hand patting the patient and provides a similar soothing effect. For example, during operation, the patting device may provide repeated application of quick, impulse-like bursts of a gentle force to the body of the patient to enable this soothing effect. This patient soothing system is intended to provide gentle patting or a massaging motion, optionally in combination with heat delivery or removal, to provide continuous comfort to patients, including, for example, newborn infants or small children.

The patient soothing system aims to solve a problem commonly encountered by nurses by soothing crying babies while the nurses are occupied. This system is intended to enable limited healthcare staff to provide continuous comfort to newborn infants or small children struggling with maladies that are eased by hugging, swaddling, gentle rubbing, or patting. For example, this system enables limited healthcare staff to provide continuous comfort to newborn infants, while not requiring constant direct involvement from the healthcare provider team. The system is able to operate unattended for short periods of time, permitting the attending nurse to step away and care for multiple infants simultaneously, while ensuring that all infants are receiving continuous comfort. As such, the system enables healthcare professionals to provide near continuous comfort to newborn infants or children that suffer from conditions such as colic, acid reflux, drug withdrawal, digestive issues, difficulties eating or sleeping, transitioning from the intrauterine environment, and so forth. The system enables operation to be customized to maximize comfort to the patient, such as time profiles for treatment or use. Additionally, while the patient soothing system is discussed herein in the context of hospital and healthcare application for infants and small children, in other embodiments, the system may be designed for use by parents or caretakers for the soothing of infants, small children, or even adults in home or healthcare settings.

One embodiment of such a system is a patient soothing system that includes a patting device configured to be pneumatically powered to rapidly inflate and deflate to provide a simulated patting force to a patient. The system has a control module including control circuitry and an air pulse regulator with at least one pneumatic solenoid valve that is fluidly connected to the patting device via a flexible tube. The control circuitry is configured to actuate the at least one pneumatic solenoid valve of the air pulse regulator to provide rapid inflation and deflation pulses of air to rapidly inflate and deflate the patting device, thereby to yield the simulated patting force.

In some embodiments, the patient is an infant, and the patient soothing system further includes a blanket having a pocket configured to receive the patting device, in which the blanket is configured to wrap around a torso of the infant to secure the patting device at a posterior side of the infant. In some embodiments, the blanket includes one or more additional pockets configured to receive a disposable warming pack or a disposable cooling pack. In some embodiments, the blanket is translucent to light at wavelengths ranging from about 460 nanometers (nm) to about 490 nm.

In some embodiments, the patting device includes an air bladder formed from excessive density polyethylene (EDPE), high-density polyethylene (HDPE), silicone, or any combination thereof. In some embodiments, the patting device is waterproof and configured to be handwashed with soap and water for reuse with another patient. In some embodiments, the patting device has a width of about 10 centimeters (cm), a length of about 15 cm, an inflated thickness of about 2.5 cm, and a deflated thickness about 1 cm or less.

In some embodiments, the control circuitry includes a microcontroller communicatively connected to one or more metal-oxide-semiconductor field-effect transistors (MOSFETs). The microcontroller is configured to provide control signals to switch the MOSFETs between an on-state and an off-state, and the MOSFETs is operably coupled to actuate the at least one pneumatic solenoid valve between operational states to rapidly inflate and deflate the patting device. In some embodiments, an air outlet of the air pulse regulator is fluidly connected to the patting device, an air inlet of the air pulse regulator is fluidly connected to an air source, and an air exhaust outlet of the air pulse regulator is open to ambient air pressure. In some embodiments, the air source includes an air supply of a medical treatment facility or vehicle, a compressed air cylinder, or an external air pump. In some embodiments, the control module includes an internal air pump, and the air source is the internal air pump. In some embodiments, the at least one pneumatic solenoid valve includes a 3/2-way pneumatic solenoid valve, and the 3/2-way pneumatic solenoid valve is fluidly connected to the air outlet, the air inlet, and the air exhaust outlet of the air pulse regulator. In some embodiments, the at least one pneumatic solenoid valve includes a first 2/2-way pneumatic solenoid valve and a second 2/2-way pneumatic solenoid valve, and the first 2/2-way pneumatic solenoid valve is fluidly connected to the air outlet and the air inlet of the air pulse regulator, and the second 2/2-way pneumatic solenoid valve is fluidly connected to the air outlet and the air exhaust outlet of the air pulse regulator.

In some embodiments, the control module includes power conversion circuitry configured to receive and convert alternating current (AC) mains electrical energy into direct current (DC) electrical energy to power at least the control circuitry and the at least one pneumatic solenoid valve of the air pulse regulator during operation. In some embodiments, the control module includes an on-board battery configured to provide DC electrical energy to power at least the control circuitry and the at least one pneumatic solenoid valve of the air pulse regulator during operation. In some embodiments, the simulated patting force is about 30 Newtons (N). In some embodiments, the simulated patting force has a patting rate ranging from about 1 hertz (Hz) to about 3 Hz. In some embodiments, the each of the inflation pulses of air and each of the deflation pulses of air have an air pulse duration ranging from about 650 milliseconds (ms) to about 275 ms.

In some embodiments, the control module includes a user interface configured to receive user inputs to configure parameters of a patting treatment, and the parameters of the patting treatment include a patting rate, a patting force, or a patting treatment duration, or any combination thereof. In some embodiments, the user interface includes a plurality of buttons and a plurality of indicator light emitting diodes (LEDs), in which the plurality of buttons is configured to receive the user inputs to define the parameters of the patting treatment, and the plurality of indicator LEDs is configured to selectively illuminate to indicate the parameters of the patting treatment.

One embodiment of such a method is a method of using a patient soothing system. The method includes fluidly connecting a control module of the patient soothing system to a patting device of the patient soothing system via a flexible tube. The method includes placing the patting device in indirect contact with a patient. The method includes activating the patting device via a user interface of the control module to initiate a patting treatment in which the patting device rapidly inflates and deflates to provide a simulated patting force to the patient.

In some embodiments, the method includes deactivating the patting device via the user interface of the control module to cease the patting treatment. In some embodiments, after conclusion of the patting treatment, the method includes removing the patting device from indirect contact with the patient, disconnecting the control module from the patting device, and washing and drying the patting device for reuse. In some embodiments, the patient is an infant, and the placing of the patting device in indirect contact with the patient includes inserting the patting device into a pocket of a blanket, and wrapping the infant with the blanket such that the patting device is positioned at a posterior side of the patient. In some embodiments, the method includes inserting a warming pack or a cooling pack into a second pocket of the blanket.

In some embodiments, prior to activating the patting device, the method includes selecting one or more patting treatment parameters for the patting treatment via the user interface of the control module, and the patting treatment parameters include a strength of the simulated patting force, a patting rate, or a patting treatment duration, or any combination thereof. In some embodiments, the providing of the simulated patting force to the patient includes repeatedly: (i) actuating at least one pneumatic solenoid valve of the patient soothing system into a first operational position to inflate the patting device, and (ii) after a predefined air pulse duration, actuating the at least one pneumatic solenoid valve of the patient soothing system into a second operational position to deflate the patting device.

In some embodiments, the simulated patting force is about 30 N. In some embodiments, the simulated patting force has a patting rate ranging from about 1 Hz to about 3 Hz. In some embodiments, the patting treatment has a patting treatment duration that ranges from about 10 minutes to about 30 minutes.

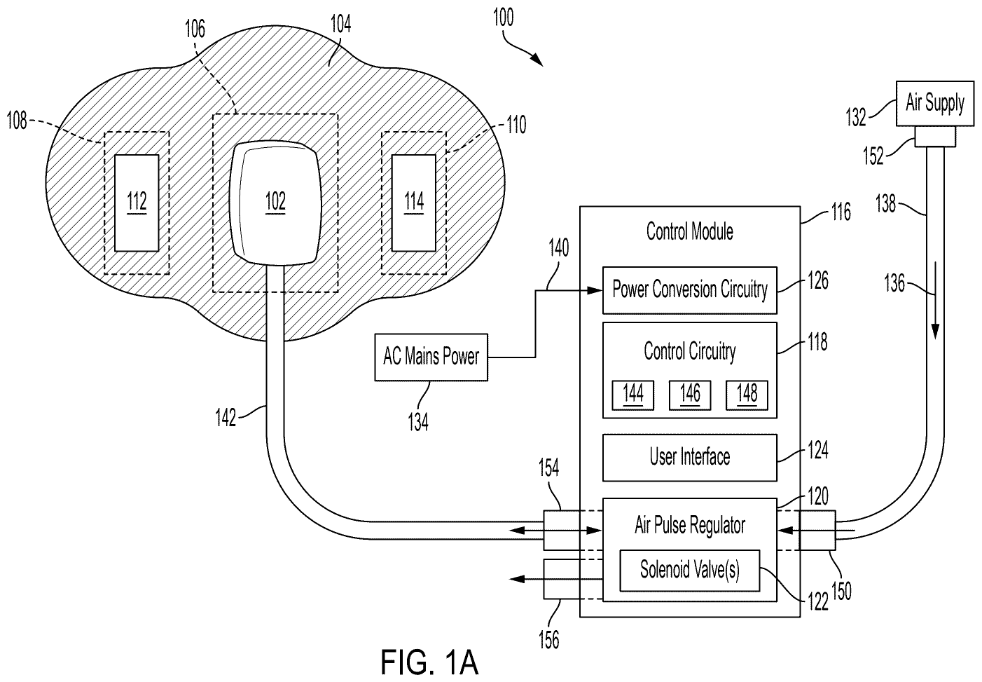

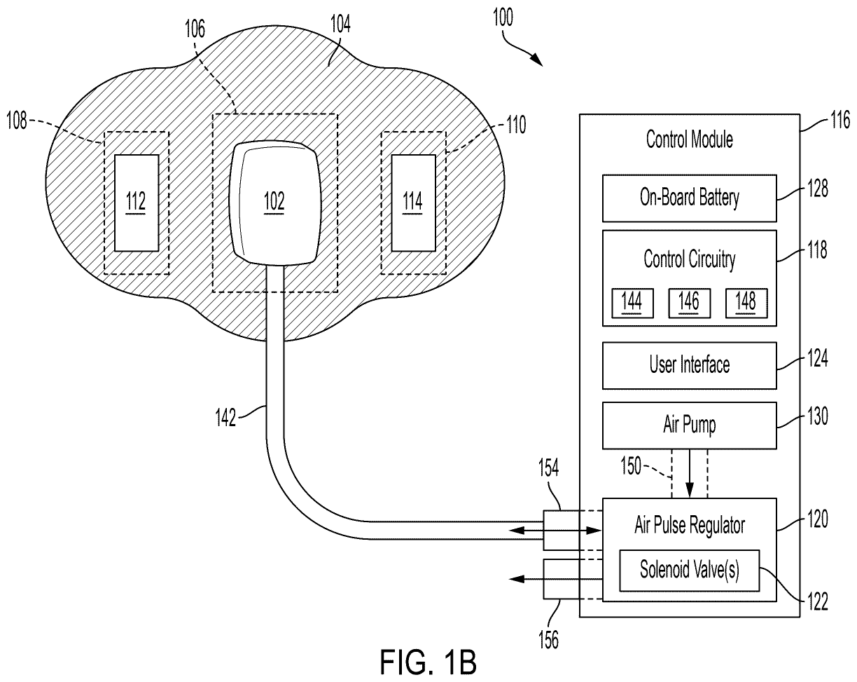

FIGS. 1A and 1B are diagrammatic representations of embodiments of a patient soothing system 100. In certain embodiments, the patient soothing system 100 is implemented as an infant soothing system designed to provide continuous comfort to a newborn infant of a size between about 5 pounds (lbs.) (2.3 kilograms (kg)) and about 10 lbs. (4.5 kg) for a predetermined duration (e.g., 30 minutes) without interruption. The embodiments of the patient soothing system 100 illustrated in FIGS. 1A and 1B include a patting device 102 (also referred to herein as a reusable mechanical stimulation module) and a wrap or blanket 104. The patting device 102 of the patient soothing system 100 may be sufficiently sized to comfortably fit within a pocket 106 of the blanket 104 in which the infant is or will be secured (e.g., wrapped, swaddled). In some embodiments, the blanket 104 of the patient soothing system 100 is a disposable cloth panel capable of wrapping the torso of an infant (e.g., a neonate, a pediatric patient). In some embodiments, the blanket 104 is further compatible with phototherapy (e.g., translucent to light of particular frequencies), such as wavelengths ranging from about 460 nanometers (nm) to about 490 nm that are used for treatment of jaundice. For the embodiments illustrated in FIGS. 1A and 1B, in addition to the pocket 106 for receiving the patting device 102, the blanket 104 also includes one or more additional pockets 108, 110 that can accept one or more warming/cooling packs 112, 114 (e.g., disposable chemical warming/cooling packs). In some embodiments, the pockets 106, 108, 110 may each be sized to accept either the warming/cooling packs 112, 114 or the patting device 102, enabling greater flexibility in how the patting device 102 and warming/cooling packs 112, 114 can be positioned on the patient. Additionally, in some embodiments, the pockets 106, 108, 110 are disposed on the outer side of the blanket 104, while the inner side is designed to contact the patient, which blocks or prevents the patient from directly contacting the patting device 102 and/or the warming/cooling packs 112, 114.

For the embodiment illustrated in FIGS. 1A and 1B, the patient soothing system 100 includes a control module 116 that is designed to be positioned on a surface (e.g., a floor or table) near the patient. Embodiments of the control module 116 may include various components, such as control circuitry 118, an air pulse regulator 120 having one or more solenoid pneumatic valves 122, and a user interface 124. In some embodiments, the control module 116 may include additional components, such power conversion circuitry 126, an on-board battery 128, and/or an internal air pump 130. For example, as illustrated FIG. 1A, in some embodiments, the control module 116 may be designed to receive external power and/or an external air supply to provide the functionality described herein. As illustrated in FIG. 1B, in some embodiments, the control module 116 may be designed to be self-contained to provide the functionality described herein without requiring external power and/or an external air supply. It may be appreciated that the embodiments of the patient soothing system 100 illustrated in FIGS. 1A and 1B are merely examples, and that in other embodiments, the patient soothing system 100 may include a different combination of components. For example, in an embodiment, the control module 116 may be fluidly connected to an external air supply 132 and include an on-board battery 128 to power the patient soothing system 100. In another example embodiment, the control module 116 may include power conversion circuitry 126 that powers the patient soothing system 100 using an external power source, and also include an internal air pump 130 of the control module 116. As such, the control module 116 and patient soothing system 100 may be customized for particular patients and/or particular treatment environments.

For the embodiment of the patient soothing system 100 illustrated in FIG. 1A, the control module 116 receives alternating current (AC) mains power 134 and receives an air flow 136 from an external air supply 132 via an air supply line 138 that is fluidly and pneumatically connected to the control module 116. For example, the electrical power supplied by AC mains 134 may be used to power various components of the control module 116, such as to power the control circuitry 118, to operate the one or more pneumatic solenoid valves 122 of the air pulse regulator 120, to operate the user interface 124, and/or to operate an internal air pump 130. The control module 116 may include power conversion circuitry 126 to convert the AC mains power 134 into direct current (DC) voltages (e.g., 12 volt, 24 volt) to power the components of the control module 116. In some embodiments, the external air supply 132 may be implemented as an external air pump that may suitably connected (e.g., fluidly connected, electrically connected, and/or communicatively connected) to the control module 116.

For the embodiment of the patient soothing system 100 illustrated in FIG. 1B, the control module 116 is powered by an on-board battery 128. For example, DC electrical power supplied by the on-board battery 128 may be used to power the control circuitry 118, to operate the one or more pneumatic solenoid valves 122 of the air pulse regulator 120, to operate the user interface 124, and/or to operate the internal air pump 130 of the control module 116. The embodiment of the patient soothing system 100 illustrated in FIG. 1B may be especially useful for treating patients in environments in which access to reliable external power and/or external air flow may be limited, such as for treating patients in developing countries, in war zones, in patient transport environments. Additionally, in terms of connections, while the embodiment of the patient soothing system 100 illustrated in FIG. 1A includes (i) a power cable 140 to electrically connect the control module 116 to AC mains power 134, (ii) an air supply line 138 to fluidly connect the control module 116 to the external air supply 132, and (iii) a flexible tube 142 that connects the control module 116 to the patting device 102, the embodiment of the patient soothing system 100 illustrated in FIG. 1B only includes the flexible tube 142. As such, the embodiment of the patient soothing system 100 illustrated in FIG. 1B may be useful for treatment environments in which the number of connections (e.g., tubes, lines, cables) needs to be minimized, for example, to ensure medical practitioners have sufficient freedom of movement in a treatment environment.

In some embodiments, the control circuitry 118 of the control module 116 includes a microcontroller unit 144 (e.g., a ATmega328 microcontroller). The control circuitry 118 may include memory 146 (e.g., random access memory (RAM), read-only memory (ROM), flash memory, solid state disk (SSD)) that stores instructions that are executed by the microcontroller unit 144 to control operation of the patient soothing system 100. In some embodiments, the memory 146 may be implemented as a component that is integrated into the microcontroller unit 144. The control circuitry 118 may include one or more power-rated metal-oxide-semiconductor field-effect transistors (MOSFETs) 148. The microcontroller unit 144 may be programmed to control the MOSFETs 148 to switch voltages on and off in order to operate the one or more pneumatic solenoid valves 122 of the air pulse regulator 120, as discussed herein. For example, based on inputs received from the user interface 124, the control circuitry 118 may provide control signals to actuate the one or more pneumatic solenoid valves 122 of the air pulse regulator 120 in accordance with predetermined patting treatment parameters (e.g., a patting rate, a patting force, and/or a patting treatment duration) of the patient soothing system 100.

The control module 116 includes an air pulse regulator 120 having a number of gas orifices (e.g., gas inlet, gas outlets) designed to receive and/or provide a gas flow at various time to operate the patting device 102, in which the orifices of the air pulse regulator 120 correspond to gas orifices (e.g., gas inlet, gas outlets) of the control module 116. The gas orifices of the control module 116 include an air inlet 150 that is designed to be fluidly and pneumatically connected to an internal or external compressed air source. For the embodiment illustrated in FIG. 1A, the air inlet 150 is an external air inlet that is fluidly and pneumatically connected to the external air supply 132 via the air supply line 138. In some embodiments, the external air supply 132 may be a compressed air supply line of a medical treatment facility or vehicle (e.g., a hospital, a clinic, an ambulance, a medical transport helicopter or airplane), a compressed air cylinder, or an external air pump. For such embodiments, the connection to the external air supply 132 may obviate the need for the control module 116 of the patient soothing system 100 to include an internal air pump 130, desirably reducing the size, complexity, manufacturing cost, power consumption, and operational noise associated with operation of the patient soothing system 100. For such embodiments, the pressure and/or flow of the compressed air may be regulated by a pressure and/or flow regulator 152 positioned at an outlet of the external air supply or at the air inlet 150 of the control module 116 to ensure that the maximum pressure is below a rupturing pressure of the patting device 102. For embodiments in which the patient soothing system 100 includes the internal air pump 130, as illustrated in FIG. 1B, the internal air pump 130 may be fluidly and pneumatically connected to an internal air inlet 150 of the air pulse regulator 120. For such embodiments, the internal air pump 130 may be rated for a maximum pressure that is below a rupturing pressure of the patting device 102.

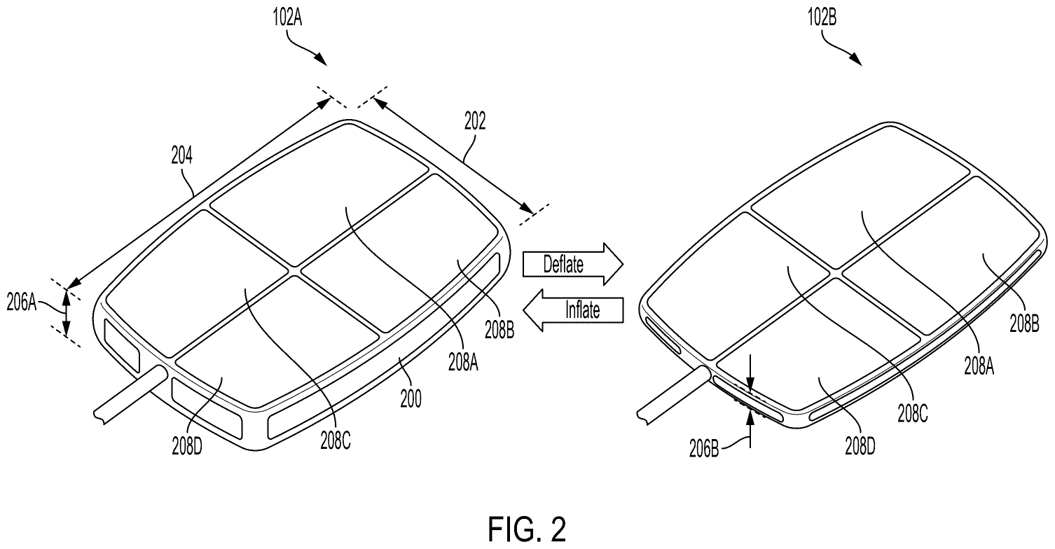

The gas orifices of the control module 116 include an air outlet 154 that is designed to fluidly and pneumatically connect to the patting device 102 via the flexible tube 142, and includes an air exhaust outlet 156 designed to be in fluid communication with ambient atmospheric pressure. The one or more pneumatic solenoid valves 122 of the air pulse regulator 120 are controlled by the control circuitry 118 to provide a pulse of air from the internal or external air supply source to at least partially inflate the patting device via the flexible tube 142. Subsequently, the one or more pneumatic solenoid valves 122 of the air pulse regulator 120 are suitably actuated to fluidly and pneumatically connect the interior of the inflated patting device 102 to the exhaust outlet 156, rapidly deflating the patting device 102. As the process repeats, the rapid inflation and deflation of the patting device 102 results in the patting motion that soothes or otherwise treats the patient, as further illustrated in FIG. 2.

In some embodiments, the control module 116 includes a user interface 124 that enables an operator to select the desired patting rate, patting duration, and/or patting force for a given treatment. In some embodiments, the user interface 124 may include buttons, dials, light emitting diodes (LEDs), a display, a touchscreen, a speaker, or other suitable input or output devices. In some embodiments, the control module 116 can be configured to provide a patting rate ranging from about 60 pats/minute or 1 hertz (Hz) to about 180 pats/minute or 3 Hz for a predetermined or selected patting treatment duration (e.g., 10 minutes, 20 minutes, 30 minutes, 45 minutes, 1 hour, or more). For example, in certain embodiments, the patting device 102 can be configured to provide a patting rate of selected from 60 pats/minute (1 Hz), 120 pats/minute (2 Hz), or 180 pats/minute (3 Hz), for a patting treatment duration that is selected in ten-minute increments (e.g., 10 minutes, 20 minutes, 30 minutes). For example, in some embodiments, the user interface 124 of the control module 116 may include buttons and indicator LEDs located on a prominent face of the control module 116 that may be used to configure the system. For example, in some embodiments, the buttons of the user interface 124 may include a power button, a “begin treatment” button, a “end treatment” button, at least one button to adjust patting rate, at least one button to adjust patting treatment duration, and/or at least one button to adjust patting force. In an example embodiment, one LED is used to indicate when the system is powered on, and three LEDs are used to indicate the current patting rate of the system (e.g., one lit LED indicates a patting rate of 1 Hz, two lit LEDs indicate a patting rate of 2 Hz, and three lit LEDs indicate a patting rate of 3 Hz).

FIG. 2 is a diagrammatic representation of an embodiment of the patting device 102 in an inflated state 102A and in a deflated state 102B. In some embodiments, the patient soothing system 100 is capable of providing a patting force of about 30 Newtons (N) to the patient via the patting device 102. The patting device 102 generally includes an air bladder 200 that is designed for rapid pneumatic inflation and deflation to provide the patting effect. For most applications, the patting device 102 is designed to be positioned posteriorly (under the back and/or bottom) of a patient in a supine position (lying on their back, face up). As such, the patting device 102 is desirably flexible and has a limited thickness to ensure that the patient is comfortable, and such that the patient's head and neck can be supported at all times. In some embodiments for infant applications, the patting device 102 has dimensions that include a width 202 of about 4 inches (in.) or 10 centimeters (cm) and a length 204 of about 6 in. or 15 cm. In the inflated state, the patting device 102A has a thickness 206A of about 1 in. or 2.5 cm, and in the deflated state, the patting device 102B has a thickness 206B of 1 cm or less. In certain embodiments, the patting device includes multiple air chambers (e.g., air chambers 208A, 208B, 208C, and 208D) to provide additional structure and reduce the risk of the air bladder being ruptured during use.

Embodiment of both the blanket 104 and the patting device 102 of the patient soothing system 100 are designed from materials that are safe for human contact and, more specifically, may be made of hypoallergenic materials to prevent undesired patient reactions. In general, the patting device is made of a waterproof material (e.g., excessive density polyethylene (EDPE), high-density polyethylene (HDPE), silicone), which enables the device to be hand washed using soap and water between uses with the same or different patients. In some embodiments, the air bladder 200 of the patting device 102 may be heat sealed. In some use cases, the patting device 102 may be fluidly and pneumatically coupled to the control module 116 via the flexible tube 142 prior to insertion into the pocket 106 of the blanket 104, while in other cases, the patting device 102 may be fluidly and pneumatically coupled to the control module 116 via the flexible tube 142 after insertion into the pocket 106 of the blanket 104. The flexible tube 142 is designed to quickly and easily be connected to and disconnected from the patting device 102.

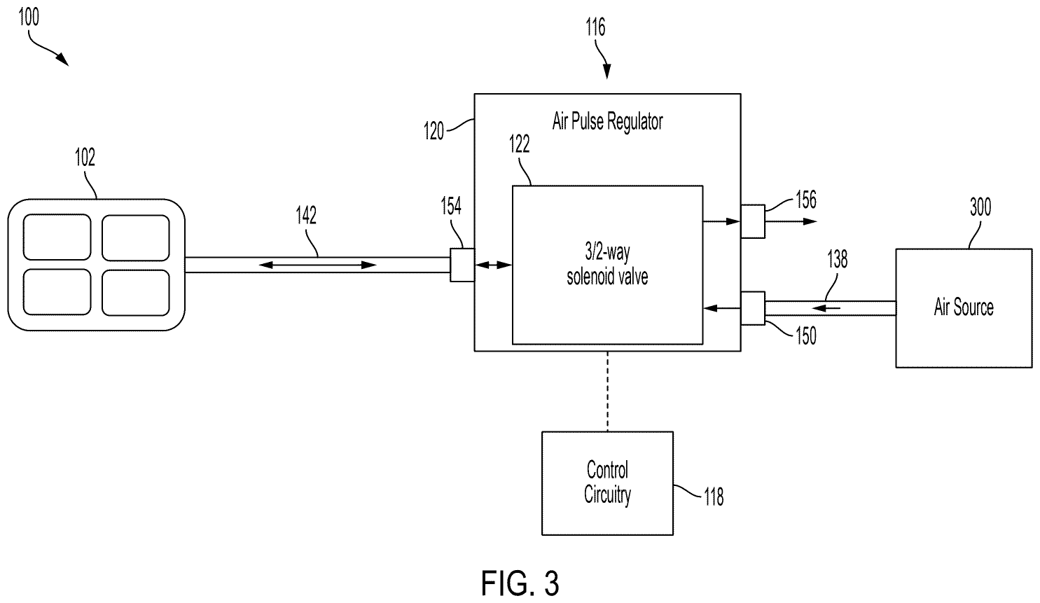

FIG. 3 is a diagrammatic representation of air flow for an embodiment of the patient soothing system 100 during operation. It should be noted that FIG. 3 illustrates components of the patient soothing system 100 that are specifically related to air flow during operation, and the patient soothing system 100 may include additional components, as discussed herein. For the embodiment illustrated in FIG. 3, the patting device 102 is fluidly or pneumatically connected to the air outlet 154 of the air pulse regulator 120. An air source 300, which may correspond to the external air supply 132 or the internal air pump 130 in different embodiments, is fluidly and pneumatically connected to the air inlet 150 of the air pulse regulator 120, while the exhaust outlet 156 of the air pulse regulator 120 that is open to ambient air pressure. For the illustrated embodiment, the air pulse regulator 120 includes a single, fast-acting 3/2-way pneumatic solenoid valve 122. The 3/2-way pneumatic solenoid valve 122 has three ports: an inlet port, an outlet port, and an exhaust port that respectively correspond to the air inlet 150, the air outlet 154, and the air exhaust outlet 156 of the air pulse regulator 120. The 3/2-way pneumatic solenoid valve 122 has two operating positions (i.e., an inlet-outlet connected position and an outlet-exhaust connected position) and is configured to switch between the two operating positions in response to control signals received from the control circuitry 118. In the inlet-outlet connected position, the 3/2-way pneumatic solenoid valve 122 fluidly and pneumatically connects the air source 300 to the interior of the patting device 102 to provide an inflation pulse that inflates the patting device. In the outlet-exhaust connected position, the 3/2-way pneumatic solenoid valve 122 fluidly and pneumatically connects the interior of the patting device 102 to the ambient air pressure at the exhaust outlet 156 to provide a deflation pulse that deflates the patting device 102. As such, the illustrated embodiment includes a single valve to both inflate and vent the air bladder of the patting device 102, which can limit the cost and complexity of manufacturing the air pulse regulator 120 and the patient soothing system 100.

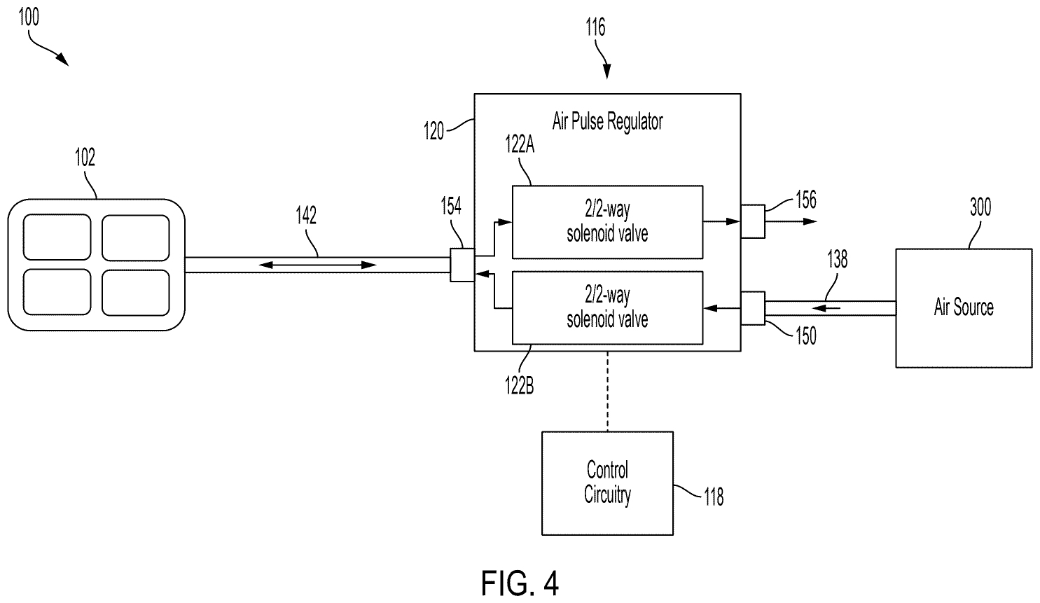

FIG. 4 is a diagrammatic representation of air flow for another embodiment of the patient soothing system 100 during operation. It should be noted that FIG. 4 illustrates components of the patient soothing system 100 that are specifically related to air flow during operation, and the patient soothing system 100 may include additional components, as discussed herein. For the embodiment illustrated in FIG. 4, the patting device 102 is fluidly or pneumatically connected to the air outlet 154 of the air pulse regulator 120. An air source 300, which may correspond to the external air supply 132 or the internal air pump 130 in different embodiments, is fluidly and pneumatically connected to the air inlet 150 of the air pulse regulator 120, while the exhaust outlet 156 of the air pulse regulator 120 that is open to ambient air pressure. For the embodiment illustrated in FIG. 4, the air pulse regulator 120 includes two fast-acting 2/2-way pneumatic solenoid valves 122A, 122B, including a first 2/2-way pneumatic solenoid valve 122A and a second 2/2-way pneumatic solenoid valve 122B. Each 2/2-way pneumatic solenoid valve 122A, 122B has two ports (i.e., an inlet and an outlet) and two operating positions (i.e., open and closed), in which the valves 122A, 122B are configured to switch between the two operating positions in response to control signals received from the control circuitry 118. To inflate the patting device 102 first 2/2-way pneumatic solenoid valve 122A is actuated to be in the closed position while the second 2/2-way pneumatic solenoid valve 122B is actuated to be in the open position to fluidly and pneumatically connects the air source 300 to the interior of the patting device 102 to provide an inflation pulse. To deflate the patting device 102, the second 2/2-way pneumatic solenoid valve 122B is actuated to be in the closed position while the first 2/2-way pneumatic solenoid valve 122A is actuated to be in the open position to fluidly and pneumatically connect the interior of the patting device 102 to the ambient air pressure at the exhaust outlet 156 to provide a deflation pulse.

In some embodiments, each of the inflation and deflation pulses may have a predetermined duration based on the instructions encoded within the control circuitry 118. In some embodiments, each of the inflation and deflation pulses have the same predetermined air pulse duration ranging from about 650 milliseconds (ms) to about 275 ms, while in other embodiments, the inflation pulses and the deflation pulses may have different predetermined air pulse durations. In some embodiments, the duration of each of the inflation and deflation pulses may vary depending on a patting rate setting received by the control module 116 via the user interface 124. For example, in an embodiment, the duration of each of the inflation and deflation pulse may be about 650 ms for a 1 Hz patting rate, about 350 ms for a 2 Hz patting rate, and 275 ms for a 3 Hz patting rate.

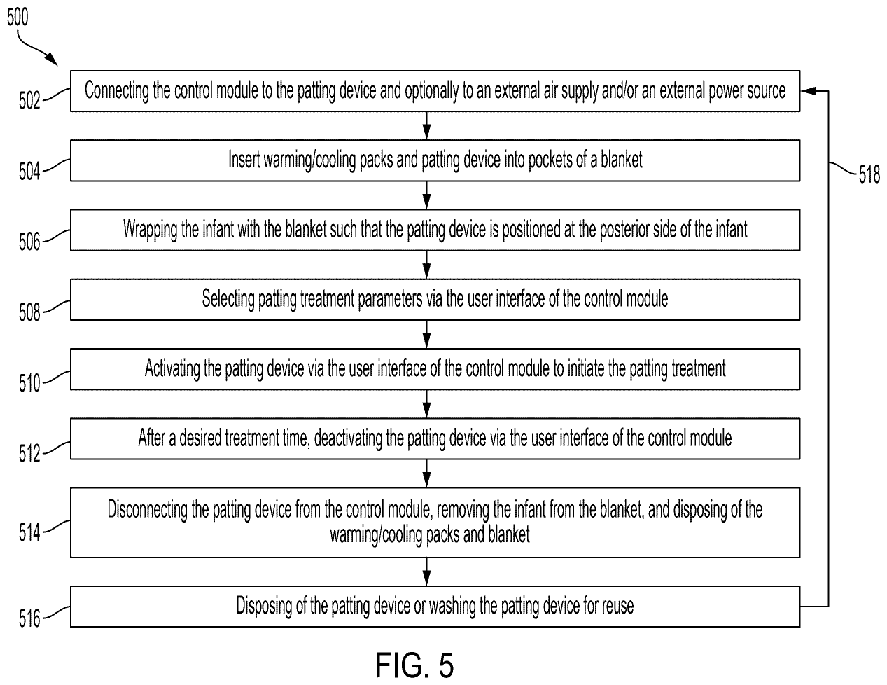

FIG. 5 is a diagrammatic representation of an embodiment of a method 500 of using the patient soothing system 100 to treat an infant or neonate patient. The method 500 is merely provided as an example, and in other embodiments, the method 500 may include additional steps, fewer steps, or repeated steps, in accordance with the present disclosure. The method 500 is discussed with reference to elements illustrated in FIGS. 1A and 1B. For the embodiment illustrated in FIG. 5, the method 500 begins in block 502 with connecting the control module 116 to the patting device 102 and optionally to an external air supply 132 and/or an external power source (e.g., AC mains power 134). As noted, the air pulse regulator 120 of the control module 116 may be fluidly and pneumatically connected to the patting device 102 via the flexible tube 142. As noted, in different embodiments, the air pulse regulator 120 of the control module 116 may be fluidly and pneumatically connected to either an external air source (e.g., external air supply 132) or an internal air source (e.g., internal air pump 130). Additionally, as noted, in different embodiments, the control module 116 may be powered by external AC mains power 134 or by the on-board battery 128.

For the embodiment illustrated in FIG. 5, the method 500 continues in block 504 with inserting the patting device 102, and optionally one or more warming/cooling packs 112, 114, into the pockets 106, 108, 110 of the blanket 104. As noted, in different embodiments, the patting device 102 may be fluidly connected to the air pulse regulator 120 of the control module 116 either before or after the patting device 102 is inserted into the pocket 106 of the blanket. The method 500 continues in block 506 with swaddling or wrapping the infant with the blanket 104 such that the patting device 102 is located at (e.g., adjacent to, proximate to, in indirect contact with) the patient's back and/or bottom. For embodiment in which the patient soothing system 100 is used to treat larger patients (e.g., pediatric or adult patients) without using the blanket 104, the patting device 102 may be placed in indirect contact with the patient (e.g., with one or more intervening layers of fabric disposed between the patting device 102 and the patient).

For the embodiment illustrated in FIG. 5, the method 500 continues in block 508 with selecting patting treatment parameters (e.g., patting rate, patting treatment duration, and/or patting pressure) via the user interface 124 of the control module 116. For example, in some embodiments, a user or medical professional may press buttons or interact with other suitable user input mechanisms of the user interface 124 to adjust the patting treatment parameters. Additionally, the user or medical professional may determine the configured patting treatment parameters based on the illumination of corresponding indicator LEDs or other suitable user output mechanisms of the user interface 124. In some embodiments, the patient soothing system 100 may alternatively be preconfigured with predefined set of patting treatment parameters (e.g., a predefined patting rate, a predefined patting treatment duration, and a predefined patting pressure).

For the embodiment illustrated in FIG. 5, the method 500 continues in block 510 with activating the patting device 102 via the user interface 124 of the control module 116 to initiate the patting treatment. For example, in some embodiments, a user or medical professional may press a button (e.g., a “begin treatment” button) of the user interface 124 to initiate the patting treatment. In some embodiments, the patient soothing system 100 may automatically cease operation at the conclusion of a predefined patting treatment duration, for example, configured or defined based on user inputs received via the user interface 124 of the control module 116. When the treatment is discontinued before the end of a predefined patting treatment duration, the method 500 continues in block 512 with deactivating the patient soothing system 100 via the user interface 124 of the control module 116. For example, in some embodiments, a user or medical professional may press a button (e.g., an “end treatment” button) of the user interface 124 to conclude the patting treatment.

For the embodiment illustrated in FIG. 5, the method 500 continues in block 514 with disconnecting the patting device 102 from the control module 116, removing the baby from the blanket 104, and disposing of the warming/cooling packs 112, 114 and the blanket 104. In some embodiments, the warming/cooling packs 112, 114 and/or the blanket 104 may be washable and reusable. The method 500 concludes in block 516 with washing the patting device 102 for reuse or disposing of the patting device 102. When the patting device 102 is washed and dried rather than being disposed, the patting device 102 is subsequently ready for reuse with a new blanket 104, as indicated by the arrow 518.

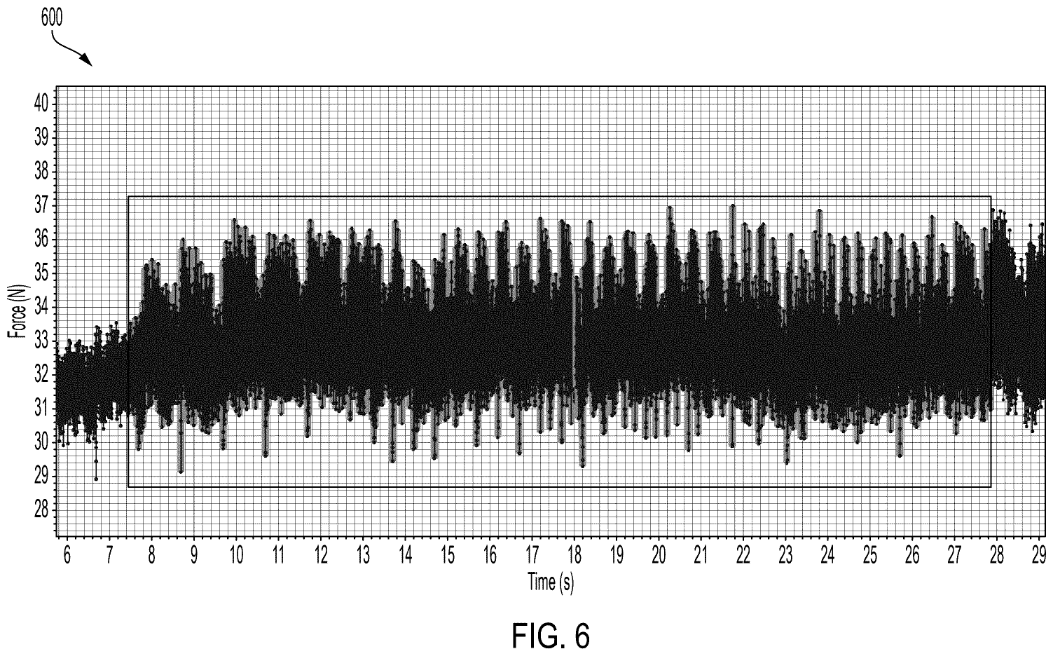

Prototype patient soothing systems were developed and tested. FIG. 6 is a diagrammatic representation of experimental data determined during testing of an example prototype. The graph 600 illustrated in FIG. 6 plots force (N) applied by the patting device over time during operation. A large noise component was observed in the force data, which is believed to be due to the vibration of the air pump operating close to the force measuring device. Despite the large noise component, the observed periodic spikes are indicative of a simulated “tap” or “pat” being applied. The patting rate was then varied through the three set speeds (i.e., 1 Hz, 2 Hz, and 3 Hz) and the force response was measured. For the prototype device, the strongest force response was observed at a speed of 3 Hz, which is believed to be due to a larger amount of air being retained within the air bladder even during the deflation part of the pulse cycle.

While the patient soothing system 100 is primarily described herein in the context of providing a patting-like sensation to sooth infants and neonates, it should be appreciated that other applications are envisioned. For example, larger embodiments of the patting device 102 (e.g., 2-4 times the dimensions discussed above) or arrays of the patting device 102 (e.g., containing 2-8 patting devices) may be used to soothe patients of larger sizes, such as larger pediatric or adult patients. For example, embodiments of the system 100 may be used to provide tactile stimulation to comatose patients and/or patients experiencing locked-in syndrome. For bedridden patients, the system 100 may desirably promote separation and/or air circulation between the patient and the bed, which may decrease the risk of bed sores. The system 100 may be used in the treatment of post-operative patients, such that the gentle patting motion promotes local blood and/or lymphatic fluid flow to aid in wound healing. In some embodiments, the system 100 may include a heating element capable of selecting warming the air delivered to the patting device 102, such that the system may be used to provide gentle massage with heating and cooling cycles over a patting treatment duration, for example, to treat a sore, strained, or pulled muscle of the patient. The system 100 may be used to provide chest percussion therapy (CPT) to loosen or dislodge mucous in the lungs of a patient. In some embodiments, the patting may be integrated within a patient's bedding (e.g., near the surface of a mattress, mat, or foam cushion), such that the patting device 102 can provide the patting motion to a patient in contact with (e.g., lying prone or supine on) the bedding. In addition to soothing patients in a medical treatment environment, such embodiments may be used in home environments, for example, to provide patting stimulation in response to detected conditions. For example, the system 100 may be implemented in tandem with (e.g., communicatively connected with) systems that detect snoring or apneic episodes in sleeping patients and selectively activated to provide mild stimulation in response to detection of snoring or an apneic episode to promote normal breathing in the sleeping patient.

Other objects, features, and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A patient soothing system comprising:
a patting device configured to be pneumatically powered to rapidly inflate and deflate to provide a simulated patting force to a patient; and
a control module including control circuitry and an air pulse regulator with at least one pneumatic solenoid valve that is fluidly connected to the patting device via a flexible tube, the control circuitry being configured to actuate the at least one pneumatic solenoid valve of the air pulse regulator to provide rapid inflation and deflation pulses of air to rapidly inflate and deflate the patting device, thereby to yield the simulated patting force.

2. The patient soothing system of claim 1, wherein the patient is an infant, and the patient soothing system further comprises a blanket having a pocket configured to receive the patting device, the blanket being configured to wrap around a torso of the infant to secure the patting device at a posterior side of the infant.

3. The patient soothing system of claim 2, wherein the blanket includes one or more additional pockets configured to receive a disposable warming pack or a disposable cooling pack.

4. The patient soothing system of claim 2, wherein the blanket is translucent to light at wavelengths ranging from about 460 nanometers (nm) to about 490 nm.

5. The patient soothing system of claim 1, wherein the patting device comprises an air bladder formed from excessive density polyethylene (EDPE), high-density polyethylene (HDPE), silicone, or any combination thereof.

6. The patient soothing system of claim 1, wherein the patting device is waterproof and configured to be handwashed with soap and water for reuse with another patient.

7. The patient soothing system of claim 1, wherein the patting device has a width of about 10 centimeters (cm), a length of about 15 cm, an inflated thickness of about 2.5 cm, and a deflated thickness about 1 cm or less.

8. The patient soothing system of claim 1, wherein the control circuitry comprises a microcontroller communicatively connected to one or more metal-oxide-semiconductor field-effect transistors (MOSFETs), the microcontroller being configured to provide control signals to switch the MOSFETs between an on-state and an off-state, and the MOSFETs being operably coupled to actuate the at least one pneumatic solenoid valve between operational states to rapidly inflate and deflate the patting device.

9. The patient soothing system of claim 1, wherein an air outlet of the air pulse regulator is fluidly connected to the patting device, an air inlet of the air pulse regulator is fluidly connected to an air source, and an air exhaust outlet of the air pulse regulator is open to ambient air pressure.

10. The patient soothing system of claim 9, wherein the air source comprises an air supply of a medical treatment facility or vehicle, a compressed air cylinder, or an external air pump.

11. The patient soothing system of claim 9, wherein the control module comprises an internal air pump, and wherein the air source is the internal air pump.

12. The patient soothing system of claim 9, wherein the at least one pneumatic solenoid valve comprises a 3/2-way pneumatic solenoid valve, wherein the 3/2-way pneumatic solenoid valve is fluidly connected to the air outlet, the air inlet, and the air exhaust outlet of the air pulse regulator.

13. The patient soothing system of claim 9, wherein the at least one pneumatic solenoid valve comprises a first 2/2-way pneumatic solenoid valve and a second 2/2-way pneumatic solenoid valve, and wherein the first 2/2-way pneumatic solenoid valve is fluidly connected to the air outlet and the air inlet of the air pulse regulator, and the second 2/2-way pneumatic solenoid valve is fluidly connected to the air outlet and the air exhaust outlet of the air pulse regulator.

14. The patient soothing system of claim 1, wherein the control module comprises power conversion circuitry configured to receive and convert alternating current (AC) mains electrical energy into direct current (DC) electrical energy to power at least the control circuitry and the at least one pneumatic solenoid valve of the air pulse regulator during operation.

15. The patient soothing system of claim 1, wherein the control module comprises an on-board battery configured to provide DC electrical energy to power at least the control circuitry and the at least one pneumatic solenoid valve of the air pulse regulator during operation.

16. The patient soothing system of claim 1, wherein the simulated patting force is about 30 Newtons (N).

17. The patient soothing system of claim 1, wherein the simulated patting force has a patting rate ranging from about 1 hertz (Hz) to about 3 Hz.

18. The patient soothing system of claim 1, wherein the each of the inflation pulses of air and each of the deflation pulses of air have an air pulse duration ranging from about 650 milliseconds (ms) to about 275 ms.

19. The patient soothing system of claim 1, wherein the control module comprises a user interface configured to receive user inputs to configure parameters of a patting treatment, and wherein the parameters of the patting treatment include a patting rate, a patting force, or a patting treatment duration, or any combination thereof.

20. The patient soothing system of claim 19, wherein the user interface comprises a plurality of buttons and a plurality of indicator light emitting diodes (LEDs), wherein the plurality of buttons is configured to receive the user inputs to define the parameters of the patting treatment, and the plurality of indicator LEDs is configured to selectively illuminate to indicate the parameters of the patting treatment.

21. A method of using a patient soothing system, the method comprising:
fluidly connecting a control module of the patient soothing system to a patting device of the patient soothing system via a flexible tube;
placing the patting device in indirect contact with a patient; and
activating the patting device via a user interface of the control module to initiate a patting treatment in which the patting device rapidly inflates and deflates to provide a simulated patting force to the patient.

22. The method of claim 21, further comprising:
deactivating the patting device via the user interface of the control module to cease the patting treatment.

23. The method of claim 21, wherein, after conclusion of the patting treatment, the method further comprises:
removing the patting device from indirect contact with the patient;
disconnecting the control module from the patting device; and
washing and drying the patting device for reuse.

24. The method of claim 21, wherein the patient is an infant, and wherein the placing of the patting device in indirect contact with the patient further comprises:
inserting the patting device into a pocket of a blanket; and
wrapping the infant with the blanket such that the patting device is positioned at a posterior side of the patient.

25. The method of claim 24, wherein the method further comprises:
inserting a warming pack or a cooling pack into a second pocket of the blanket.

26. The method of claim 21, wherein, prior to activating the patting device, the method comprises:
selecting one or more patting treatment parameters for the patting treatment via the user interface of the control module, wherein the patting treatment parameters comprise a strength of the simulated patting force, a patting rate, or a patting treatment duration, or any combination thereof.

27. The method of claim 21, wherein the providing of the simulated patting force to the patient comprises repeatedly:
actuating at least one pneumatic solenoid valve of the patient soothing system into a first operational position to inflate the patting device; and
after a predefined air pulse duration, actuating the at least one pneumatic solenoid valve of the patient soothing system into a second operational position to deflate the patting device.

28. The method of claim 21, wherein the simulated patting force is about 30 Newtons (N).

29. The method of claim 21, wherein the simulated patting force has a patting rate ranging from about 1 hertz (Hz) to about 3 Hz.

30. The method of claim 21, wherein the patting treatment has a patting treatment duration that ranges from about 10 minutes to about 30 minutes.
